(19) United States
(12) Patent Application Publication
(10) Pub. No.: US 2025/0104229 A1
(43) Pub. Date: Mar. 27, 2025

(54) INTEGRATED INTRAOCCULAR NAVIGATION SYSTEM FOR OPHTHALMIC SURGERY

(71) Applicant: Alcon Inc., Fribourg (CH)

(72) Inventor: Paul R. HALLEN, Colleyville, TX (US)

(21) Appl. No.: 18/830,335

(22) Filed: Sep. 10, 2024

Related U.S. Application Data
(60) Provisional application No. 63/584,491, filed on Sep. 21, 2023.

(51) Int. Cl. G06T 7/00; A61B 34/20; A61B 34/30; G06T 15/00

(52) U.S. Cl. CPC G06T 7/0012; A61B 34/30; G06T 15/00; A61B 2034/2057; G06T 2207/10101; G06T 2207/30041

(57) ABSTRACT

A system includes an imaging device configured to perform three-dimensional imaging of at least a portion of an eye of a patient, such as one or both of an OCT and a 3D camera. A sensor is configured to sense a location of a trocar cannula positioned in the eye of the patient. A controller is configured to receive one or more three-dimensional images from the imaging device and receive coordinates of the trocar cannula from the sensor. The controller generates a three-dimensional map of the eye from the one or more three-dimensional images and the coordinates, the three-dimensional map including a representation of the trocar cannula. The controller further generates guidance for performing an ophthalmic procedure according to the three-dimensional map, such as an instrument envelope and/or instrument path. The controller outputs the guidance to a display device or uses the guidance to control a robotic arm.

### CROSS-REFERENCE TO RELATED APPLICATIONS

This application claims priority to U.S. Provisional Application No. 63/584,491, filed on Sep. 21, 2023, which is hereby incorporated by reference in its entirety.

### BACKGROUND

The present disclosure relates generally instruments used for ophthalmic treatments, particularly those used for performing vitrectomy, treating cataracts, treating glaucoma by reducing intraocular pressure (IOP), or other ophthalmic treatments.

The anatomy of the eye is very small and movements performed with instruments when performing ophthalmic surgery must be correspondingly precise. For manual surgery, visualization during surgery may be provided using an ophthalmic microscope, which may provide three-dimensional visualization of the interior of the patient's eye, including visualization instruments inserted within the eye.

It would be an advancement in the art to facilitate the visualization of the interior of an eye undergoing an ophthalmic treatment.

### SUMMARY

In certain embodiments, a system includes an imaging device configured to perform three-dimensional imaging of at least a portion of an eye of a patient. A sensor is configured to sense a location of a trocar cannula positioned in the eye of the patient. A controller is configured to receive one or more three-dimensional images from the imaging device and receive coordinates of the trocar cannula from the sensor. The controller generates a three-dimensional map of the eye from the one or more three-dimensional images and the coordinates, the three-dimensional map including a representation of the trocar cannula. The controller further generates guidance for performing an ophthalmic procedure according to the three-dimensional map. The controller is configured to at least one of (a) output the guidance to a display device and (b) control an actuator coupled to a surgical instrument within the trocar cannula according to the guidance.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example system for providing intraocular navigation in accordance with certain embodiments.

FIG. 2 is cross-sectional view of the eye with instruments and trackable cannula positioned therein in accordance with certain embodiments.

FIGS. 3A and 3B illustrate a trackable cannula in accordance with certain embodiments.

FIG. 4 is a schematic diagram of components and data for providing intraocular navigation in accordance with certain embodiments.

FIG. 5 is a process flow diagram of a method for performing intraocular navigation in accordance with certain embodiments.

FIG. 6 illustrates an example computing device that implements, at least partly, one or more functionalities for providing guidance during an ophthalmic surgery using the illuminated ophthalmic surgical instrument in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

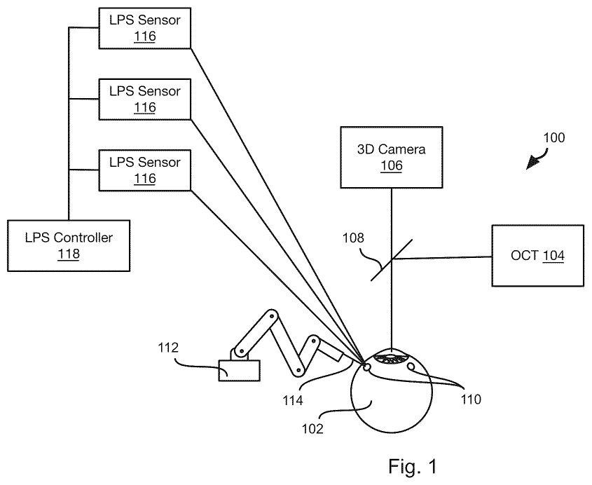

FIG. 1 illustrates an example system 100 for providing intraocular navigation. The system 100 includes one or more three-dimensional imaging devices, such as at least two three-dimensioning imaging devices, for imaging an eye 102 of a patient undergoing an ophthalmic treatment. In the illustrated embodiments, the system 100 includes an optical coherence tomography (OCT) imaging device 104 (“the OCT 104”) and a three-dimensional (3D) camera 106. The camera 106 may be a visible light and/or infrared light camera. For example, the camera 106 may be implemented as the NGENUITY 3D VISUALIZATION SYSTEM provided by Alcon Inc. of Fort Worth Texas. Other types of imaging devices may be used such as a scanning laser ophthalmoscope (SLO), fundus auto fluorescence (FAF) imaging device, multi-or hyper-spectral imaging device, or other type of imaging device. In the following description, reference is made to the OCT 104 and the camera 106 with the understanding that any of the above-referenced imaging devices may be used in a like manner.

The OCT 104 and the camera 106 may be used at separate times or used simultaneously to image the eye 102. For example, a beam splitter 108 may be used to permit light reflected from the eye 102 to reach both the camera 106 and the OCT 104.

For some ophthalmic treatments, one or more trocar cannula 110 are placed in the eye 102, such as in the sclera of the eye 102. For example, the trocar cannula 110. The trocar cannula 110 provides an entry point for instruments and helps resist damage to tissue of the eye 102 and provides a seal to resist entry of contaminants into the eye 102. For some treatments, one more additional trocar cannula 110 are provided to receive a light source and/or infusion of saline.

In some embodiments, an ophthalmic treatment is performed by or with the aid of a robotic arm 112 holding a surgical instrument 114. The robotic arm 112 may be remotely controlled by a human operator or execute a predefined treatment plan. The robotic arm 112 may dock with the trocar cannula 110 through which the instrument 114 is inserted. The robotic arm 112 may be embodied as a serial robotic arm having five or more degrees of freedom. In other embodiments, the instrument 114 is mounted to a handpiece held by a surgeon.

In some embodiments, the 3D location of each trocar cannula 110 is detected before and/or during an ophthalmic treatment. The trocar cannula 110 is may be sensed using one or more local positioning system (LPS) sensors 116 coupled to an LPS controller 118. The trocar cannula 110, LPS sensors 116, and LPS controller 118 may be implemented in various ways.

In a first example, the trocar cannula 110 includes one or more fiducial markers or other markings affixed thereto and the LPS sensors 116 are embodied as two or more cameras with the trocar cannula 110 in the field of view thereof. The locations of representations of the fiducial markers are detected in images from the LPS sensors 116 and are used to determine the location and orientation of the trocar cannula 110.

In a second example, the trocar cannula 110 includes a transmitter, such as a radio frequency identifier (RFID) transmitter or other type of transmitter. The LPS sensors 116 detect the signals from the transmitter and use the signals to determine the 3D position of the trocar cannula 110 and possibly an orientation thereof. In the second example, the transmitter and LPS sensors 116 may be embodied using any approach for detecting the position and possibly orientation of handheld controllers in a virtual reality system or other LPS approach known in the art.

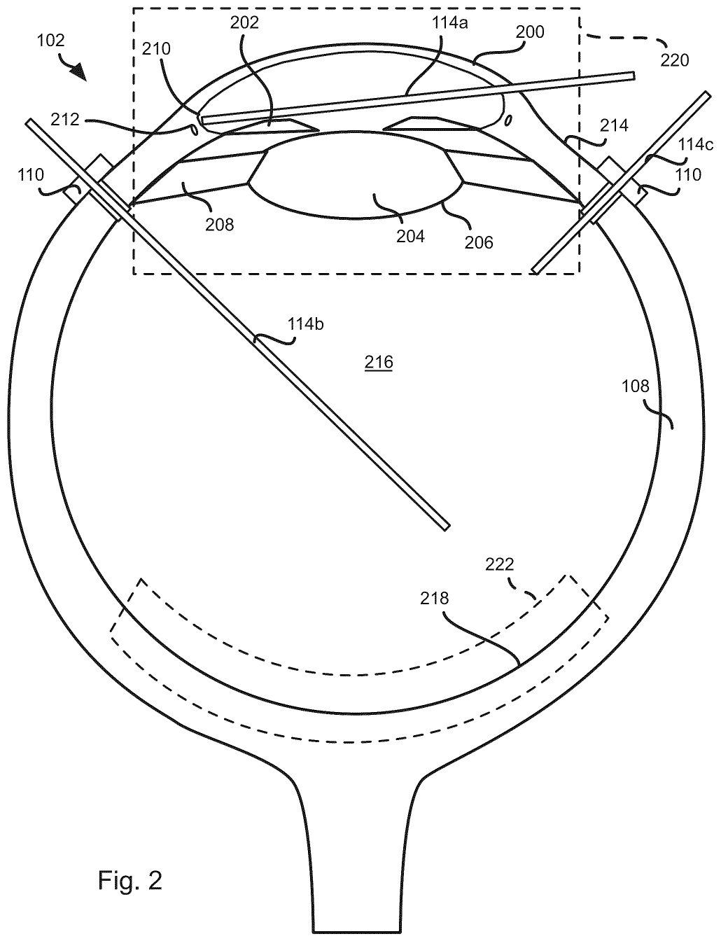

FIG. 2 is cross-sectional view of the eye 102 showing example positioning of various surgical instruments 114a, 114b, 114c and trackable 110 positioned therein. The eye 102 includes the transparent and spherical cornea through which light enters the eye 102. The light passes through the pupil defined by the iris 202. The light then passes through the crystalline lens 204, which is contained within the capsular bag 206. The capsular bag 206 is coupled to the ciliary body 208, which includes muscles for pulling on the capsular bag 206 to change the shape of the lens 204. The region defined by the cornea 200, iris, lens 204, and ciliary body 208 is known as the anterior chamber and is occupied by a fluid known as the aqueous humor. The aqueous humor is filtered through the trabecular meshwork 210 at the perimeter of the anterior chamber and into Schlemm's canal 212. Some glaucoma treatments improve drainage of the aqueous humor by placing an incision or shunt in the trabecular meshwork 210, such as using the illustrated instrument 114a inserted through the limbus, possibly through a trocar cannula 110. The limbus is defined as the junction between the cornea 200 and the sclera 214.

Ophthalmic treatments for treating cataracts, which includes removing and replacing the lens 204, may likewise use an instrument 114c inserted through the limbus, possibly through a trocar cannula 110, in order to remove the lens 204 (phacoemulsification) and to place an intraocular lens (IOL).

Light passes through the lens 204 and passes through a transparent gel known as the vitreous 216 and is incident on the retina 218, which includes light sensitive cells, i.e., the rods and cones. The region between the lens 204 and retina 218 is known as the posterior chamber. Many disorders, such as floaters and some disorders of the retina 218, are treated by vitrectomy, in which an instrument 114b is inserted through a first trocar cannula 110 and both cuts the vitreous 216 and suctions the vitreous 216 out of the posterior chamber. One or more additional instruments 114c inserted through one or more second trocar cannulas 110 may provide light to guide the procedure, infusion fluid (e.g., saline) or perform other functions.

Ophthalmic treatments to repair the retina 218, such as to perform reattachment of the retina 218, may use a similar arrangement of cannulas 110 receiving an instrument 114b for grasping and manipulating the retina 218 and an instrument 114c to provide illumination.

The OCT 104 may be used to visualize some or all of the eye 102. For example, the OCT 104 may be configured to generate a 3D image of the illustrated region 220 including the anterior chamber and possibly a portion of the posterior chamber. The OCT 104 may also be configured to generate a 3D image of region 222 including some or all of the retina 218 as well as a portion of the vitreous 216 adjacent the retina 218. The OCT 104 may penetrate some distance below the retina 218 thereby enabling visualization of various layers of the retina 218. In some implementations, the OCT 104 is configured to image the entire eye from the cornea to a depth below the surface of the retina 218. The OCT 104 may image a portion of the eye 102 extending outwardly from an optical axis of the cornea 200 and lens 204 and possibly the entire globe of the eye 102. The surface of the retina 218 may also be visualized through the cornea 200 and lens 204 using the camera 106.

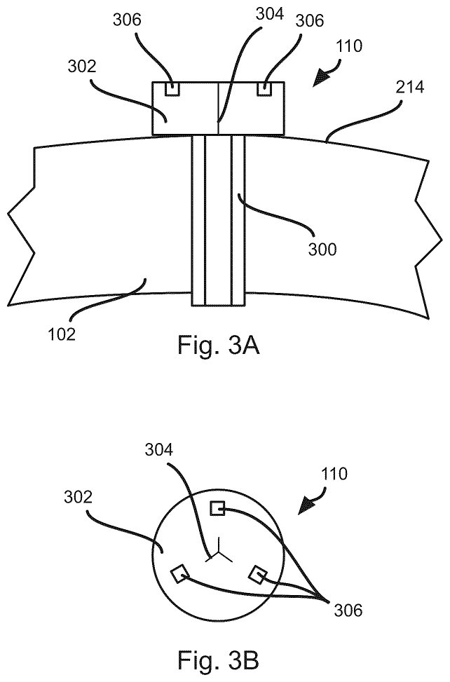

FIGS. 3A and 3B illustrate an example implementation of a trackable trocar cannula 110. The trocar cannula 110 includes a cannula 300, which is a hollow tube that passes through the globe of the eye 102 to the posterior chamber and provides a passage through which an instrument may be inserted into the posterior chamber. The cannula 300 is fastened to a head 302 of the trocar cannula 110 may extend outwardly from the cannula 300 and rest on the outer surface of the eye. The head 302 may define a valve 304 that permits insertion of an instrument through the head 302 and cannula 300 while resisting entry of contaminants into the posterior chamber and resisting leakage of fluid out of the posterior chamber. The valve 304 may be defined as slits formed in the head 302, which may be made of a flexible polymer that can be deformed upon insertion of an instrument through the valve 304.

The head 302 and/or cannula 300 have one or more trackers 306 secured thereto or formed thereon. For example, three trackers 306 may permit determining both the location and the orientation of the trocar cannula 110 where as a single tracker 306 may enable only determining the location. Each tracker 306 may be embodied as a static visual indicator that is visible in visible or infrared wavelengths. A tracker 306 embodied as a visual indicator may have features and an extent such that the detection of the tracker 306 enables determining both the location and the orientation of the trocar cannula 110. Each tracker 306 may be embodied as a transmitter transmitting a recognizable signal. For example, each tracker 306 may be embodied as a radio frequency identifier (RFID) tracker that emits a code in response to receiving an excitation signal. The tracker 306 may include, or be coupled to a power source, that supplies power to a transmitter that outputs a signal having a known frequency, encoding, and possibly timing. Emission of a signal by the transmitter may be spontaneous or in response to a control signal received by the tracker 306, such as from an LPS sensor 116 or LPS controller 118.

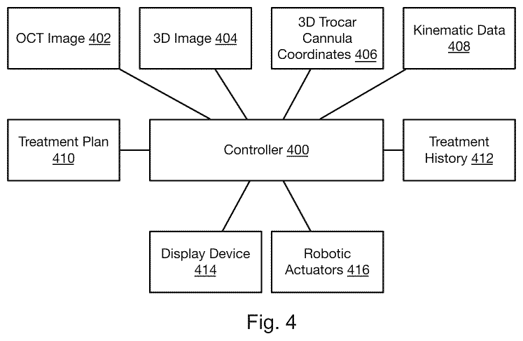

FIG. 4 is a schematic diagram of components and data for providing intraocular navigation. A controller 400 receives data before and/or during an ophthalmic treatment. The data may include one or more OCT images 402 from the OCT 104, one or more 3D images 404 from the 3D camera 106, 3D coordinates 406 of one or more trocar cannulas 110 from the LPS controller 118, kinematic data 408 describing a state of the robotic arm 112. The kinematic data 408 may include the state of each joint of the robotic arm 112 including some or all of angular position, velocity, and acceleration.

The controller 400 may further receive a treatment plan 410. The treatment plan 410 may define locations for placing shunts or incisions in the trabecular meshwork 210. The treatment plan 410 may specify a path to be followed by an end of the instrument 114a to remove the lens 204. The treatment plan 410 may specify a path to be followed by the end of an instrument 114b to remove the vitreous 216. The treatment plan 410 may specify actions to be performed using an instrument 114b to reattach a retina or perform other repairs to a retina 218. The treatment plan 410 may be defined with respect to anatomy of the eye 102 and may be automatically generated based on three-dimensional geometry of the eye 102 determined using the OCT 104 and possibly the 3D camera 106.

The controller 400 may maintain a treatment history 412. The treatment history may include a record of where the instrument 114a, 114b has been located in order to identify areas of the eye 102 that have already been treated, such as portions of the lens 204 that have been traversed by an instrument 114a configured to perform phacoemulsification or portions of the posterior chamber that have been traversed by an instrument 114b configured to perform vitrectomy.

The controller 400 may be coupled to a display device 414 and display information thereon, such as a representation of the eye 102 obtained from the OCT images 402 and 3D images 404. The representation of the eye 102 may have information superimposed thereon, such as labeling of anatomy, lines, shading, or coloring describing areas remaining to be treated according to the treatment plan 410 and the treatment history 412, areas already treated according to the treatment plan 410, or other information.

The controller 400 may be coupled to actuators 416 of the robotic arm 112 in order to control movement of the robotic arm 112 to perform automated actions according to the treatment plan 410 and the current state of the eye 102 obtained using the OCT image 402 and possibly 3D image 404.

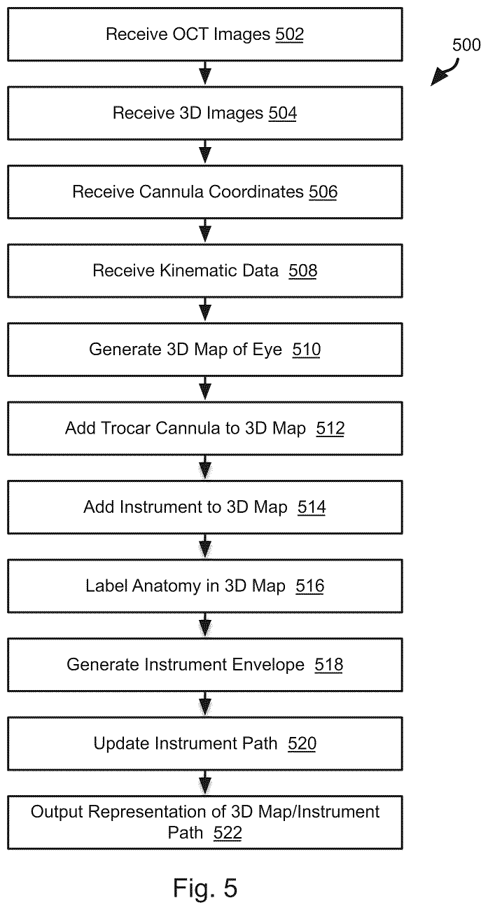

FIG. 5 is a process flow diagram of a method 500 for performing intraocular navigation. The method 500 may be performed by the controller 400 or other computing device. The method 500 may be performed prior to performing an ophthalmic treatment and may be performed throughout an ophthalmic treatment, such as according to a fixed period, in response to detected events (e.g., start of a step of the treatment plan 410, movement of the eye 102, etc.), or in response to an instruction from a surgeon.

The method 500 includes receiving, at step 502, OCT images 402 from the OCT 104. The OCT images 402 may be a series of images representing different imaging planes within the eye 102 that constitute a volumetric image of the eye 102 or a region 220, 222 within the eye at a point in time. The OCT images 402 may include one or more images of the region 220, one or more images of the region 222, or one or more images representing both regions 220, 222 possibly up to and including the entire globe of the eye.

The method 500 includes receiving, at step 504, one or more 3D images 404 from the 3D camera 106. The one or more 3D images 404 may be a set of stereoscopic images obtained using the 3D camera 106 at a point in time, a three-dimensional scene obtained from stereoscopic images, a point cloud, or other representation of a 3D surface or volume images using the 3D camera 106.

The method 500 includes receiving, at step 506, 3D coordinates 406 for one or more trocar cannulas 110. The coordinates may include coordinates for a point in 3D space on each trocar cannula of the one or more trocar cannulas 110 and possibly an indication of the orientation of each trocar cannula, in the form of coordinates for a second point on each trocar cannula or values for one, two, or three angles relative to axes defined for the 3D space. For example, the trocar cannula 110 that receives an instrument 114a that is moved relative to the eye may shift during a surgery. Accordingly, the 3D coordinates 406 may include the coordinates of the trocar cannula 110 through which the instrument 114a. Coordinates for other trocar cannulas may be omitted or may likewise be received at step 506.

The method 500 includes receiving, at step 508, kinematic data 408 describing the state of the robotic arm 112.

The OCT images 402, 3D images 404, 3D trocar cannula coordinates 406, and kinematic data 408 for an iteration of the method 500 may correspond to a substantially same moment in time, e.g., captured within 1 second, 0.1 seconds, or 10 milliseconds of one another. In other embodiments, some of steps 502-508 are performed more frequently than others. For example, kinematic data 408 may be refreshed at a much higher frequency since the robotic arm 112 is being moved with great precision. In contrast, images 402, 404 and trocar cannula coordinates 406 may be obtained less frequently than the kinematic data 408 since the eye 102 is relatively, though not necessarily completely, immobile during an ophthalmic treatment. Accordingly, one or more iterations of the method 500 may be performed without again performing some or all of steps 502-508 where data from a previous iteration of some or all of steps 502-508 is reused.

The method 500 may include generating, at step 510 a 3D map of the eye 102. Step 510 may include combining OCT images 402 for different regions 220, 222 into a single 3D OCT image. Step 510 may therefore include transforming coordinates within a 3D images 402 for regions 220, 222 into a common coordinate system and combining the transformed images for the regions 220, 222 into a single 3D image referenced herein as the 3D map. Step 510 may include adding information from a 3D image 404. For example, the voxels of the images 402 may be monochrome. Voxels the 3D map having a corresponding voxel in the 3D image 404 may be assigned the color of the corresponding voxel.

The method 500 may include adding, at step 512, representations of one or more trocar cannulas to the 3D map according to the coordinates received at step 506. For example, a 3D model of the cannula may be rendered at a location and possibly orientation in the 3D map (either directly or in a separate overlay) indicated by the coordinates received at step 506. As used herein, a separate overlay may be understood as a 3D image in which voxels corresponding to a feature represented by the separate overlay are non-zero. Where an orientation is not available, the 3D model may be oriented with the cannula 300 oriented normal to a point indicated by the coordinates and the head 302 placed flush on a surface of the eye 102.

The method 500 may include adding, at step 514, one or more representations of one or more instruments 114a, 114b, 114c to the 3D map. Step 514 may include using the kinematic data of step 508 and known dimensions of the one or more instruments 114a, 114b, 114c to render a 3D model of each of the one or more instruments 114a, 114b, 114c (e.g., at least a model of the portion protruding into the eye) in the 3D map, either directly or as a separate overlay.

Note that the representation of instruments at step 514 may be used to update the treatment history 412. In particular, a region in the 3D map corresponding to a tip of the instrument may be added to the treatment history 412 to indicate that that region has been treated. For example, the region may be a portion of the lens 204 or vitreous 216 that has been removed. The region may also be a point within the trabecular meshwork 210 in which a shunt has been placed or an incision has been made.

The method 500 includes labeling, at step 516, one or more items of anatomy in the 3D map. Step 516 may include labeling anatomy of the eye 102 in the 3D map, such as any of the items of anatomy labeled in FIG. 2 as discussed above. For the anterior chamber, additional items of anatomy may include the ciliary body band, pigmented trabecular meshwork, and the non-pigmented trabecular meshwork, to facilitate placement of incisions or shunts for treating glaucoma.

Other items of anatomy that may be labeled may include layers of the eye 102, including some or all of the inner limiting membrane, nerve fiber layer, ganglion cell layer, inner plexiform layer, inner nuclear layer, middle limiting membrane, outer plexiform layer, outer nuclear layer, external limiting membrane, retinal pigment epithelium (RPE), Bruch's membrane, choroid, or groups of adjacent layers of any of the above-referenced layers. Features of the retina may be identified such as the optic disk, avascular zone, fundus, capillaries, pathologic membranes (e.g., epiretinal membrane (ERM)), fovea, or other feature of the retina. The items of anatomy may include features of the patient's face, such as nose, eyebrows, or other features of the eye that enable the identification of the nasal and lateral sides of the eye. Accordingly, representation of trocar cannulas 110 added to the 3D map may be labeled as being on the nasal or lateral side of the eye.

Step 516 may be performed using one or more machine learning models trained to perform the task, a machine vision algorithm, registering with respect to a previously labeled 3D image, or other approach. A machine learning model for identifying an item of anatomy may be trained with images labeled with that item of anatomy and there may be multiple machine learning models trained to identify multiple items of anatomy. The labeling of step 516 may include generating an overlay, in which corresponding voxels are labeled as corresponding to a particular item of anatomy. For example, non-zero pixels in an overlay corresponding to an item of anatomy indicate voxels of the 3D map that are estimated to correspond to the item of anatomy. The labeling of step 516 may include assigning values to the voxels of the 3D map itself. There may be a single overlay or separate overlays for each item of anatomy or groups of items of anatomy that are labeled.

The above listed examples of anatomy that may be labeled is exemplary only and any other item of anatomy that can be perceived using the OCT 104 and/or 3D camera 106 may be labeled.

The method 500 may include generating, at step 518, an instrument envelope. The instrument envelope may correspond to a volume of the eye 102 on which an instrument 114a, 114b is permitted to be located.

For example, in the case of glaucoma surgery, the instrument envelope may include the anterior chamber. The instrument envelope may be defined by the anatomy labeled at step 516 and may be derived therefrom, such as a surface offset from an item of anatomy used to define the instrument envelope. For example, for glaucoma surgery, the instrument envelope may be offset from the capsular bag 206 and iris 202 that should not interact with the instrument 114a.

In the case of cataract surgery, the instrument envelope may include the interior of the capsular bag 206 as well as a portion of the capsular bag 206 adjacent the iris where an opening is formed. The instrument envelope may exclude features such as the iris 202 and ciliary body 208.

In the case of a vitrectomy, the instrument envelope may include a volume within the posterior chamber with a surface of the volume offset from the retina 218, capsular bag 206, ciliary body 208, and choroid layer by a safety margin, e.g., at least 0.1 mm. The instrument envelope may exclude sensitive items of anatomy such as blood vessels, the optic disc, fovea, or other items of anatomy.

The examples above are exemplary only. Other instrument envelopes may be defined for other ophthalmic treatments based on the necessary movement of the instrument used.

The method 500 may include updating, at step 520, an instrument path. Step 520 may take into account some or all of the treatment plan 410, treatment history 412, and the instrument envelope from step 518. The instrument path may be generated by a machine learning model trained to perform that task. For example, the machine learning model may take as inputs the 3D map and possibly the treatment plan 410, treatment history 412, or some other input. For example, an instrument path may include a path to align an instrument 114a, 114b, 114c with a cannula 110 and insert the instrument 114b, 114c into the cannula 110. Similarly, during an ophthalmic treatment, it may be necessary to move an instrument 114b, 114c to a different cannula 110. For example, an instrument path may include withdrawing an instrument 114a embodied as a phaco-vit tool from a first cannula 110, aligning the instrument 114a with a second cannula 110, and inserting the phaco-vit tool into the second cannula 110 in order to remove portions of the vitreous that are not accessible through the first cannula. In a like manner, an instrument 114b providing infusion fluid may be moved from the second cannula 110 to the first cannula.

In the case of glaucoma surgery, the instrument path may include points on the trabecular meshwork 210 defined in the treatment plan that have not yet received a shunt or incision. The points on the trabecular meshwork 210 may be defined with respect to the 3D map, i.e., account for any movement on the eye. In particular, the points on the trabecular meshwork 210 may be defined with respect to anatomy labeled at step 516 for the version of the 3D map of the current iteration of the method 500.

In the case of cataract surgery, the instrument path may include points within the instrument envelope as determined at step 518 for the capsular bag 206 that have not yet been traversed by an instrument 114a configured to perform phacoemulsification. The instrument path may traverse a path intended to efficiently traverse the volume of the capsular bag, such as regular rows, spirals or other shape.

In the case of a vitrectomy, the instrument path may include points within the instrument envelope as determined at step 518 for the posterior chamber that have not yet been traversed by an instrument 114b configured to perform vitrectomy. The instrument path may traverse a path intended to efficiently traverse the volume of the posterior chamber, such as regular rows, spirals or other shape.

In the above examples, the regions recorded in the treatment history 412 that have been previously treated may be transformed (rotated and/or translated) according to rotation, translation, and/or deformation of the eye 102. Any rotation, translation, and/or deformation may be determined by comparing changes in locations of anatomy identified at step 516 from one iteration of the method 500 to the next. Likewise, changes in the coordinates of the trocar cannula 110 from step 506 from one iteration of the method 500 to the next may be used as to estimate of rotation and/or translation of the eye 102.

In another example, an instrument path defines the location of peeling steps for peeling a membrane (e.g., ILM or ELM) from the retina, such as a spiral or zig-zag shape. The portion of the retina to be peeled may be identified based on anatomy, e.g., the pigmented area of the fovea and the avascularized zone from which the membrane is to be peeled.

In another example, the instrument path defines the path of a treatment laser emitting laser pulses for performing retinal reattachment. The path may be selected to avoid sensitive items of anatomy, such as blood vessels and anatomy identified at step 516. For example, the path may extend around the macula to avoid damaging the macula.

The method 500 may include outputting, at step 522, a representation of any of the steps of the preceding steps of the method 500. For example, a representation of the 3D map may be displayed, including a representation of overlays representing one or more trocar cannulas, one or more instruments 114a, 114b, instrument envelope, and instrument path. In the case of a human surgeon rather than a robotic arm 112, step 522 may be performed to provide guidance to the surgeon. In particular, a representation of the instrument envelope may facilitate avoidance of tissue that is not to be treated. A representation of the instrument path may help the surgeon traverse the volume to be treated as efficiently as possible. In the case of a human surgeon, the instrument path may be represented as a region to be treated rather than a specific path to be followed by an instrument 114a, 114b.

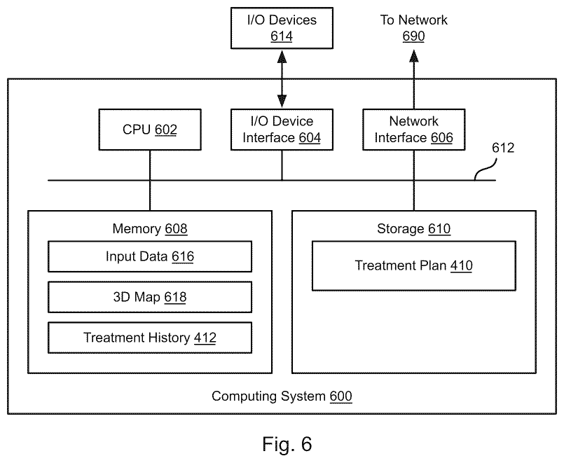

FIG. 6 illustrates an example computing system 600. The OCT 104, 3D camera 106, LPS controller 118, and controller 400 may have some or all of the attributes of the computing system 600.

As shown, computing system 600 includes a central processing unit (CPU) 602, one or more I/O device interfaces 604, which may allow for the connection of various I/O devices 614 (e.g., keyboards, displays, mouse devices, pen input, etc.) to computing system 600, network interface 606 through which computing system 600 is connected to network 690, a memory 608, storage 610, and an interconnect 612.

CPU 602 may retrieve and execute programming instructions stored in the memory 608. Similarly, CPU 602 may retrieve and store application data residing in the memory 608. The interconnect 612 transmits programming instructions and application data, among CPU 602, I/O device interface 604, network interface 606, memory 608, and storage 610. CPU 602 is included to be representative of a single CPU, multiple CPUs, a single CPU having multiple processing cores, and the like.

Memory 608 is representative of a volatile memory, such as a random access memory, and/or a nonvolatile memory, such as nonvolatile random access memory, phase change random access memory, or the like. As shown, memory 608 may store input data 616 used according to the method 500, including some or all of the one or more OCT images 402, one or more 3D images 404, 3D trocar cannula coordinates 406, and kinematic data 408. The memory 608 may further store the 3D map generated according to the method 500, which may include storing any overlays generated according to the method 500 and possibly the 3D map 618 and any overlays from one or more preceding iterations of the method 500. The memory 608 may further store the current treatment history 412 as updated during performance of an ophthalmic treatment.

Storage 610 may be non-volatile memory, such as a disk drive, solid state drive, or a collection of storage devices distributed across multiple storage systems. Storage 610 may optionally store the treatment plan 410.

In a first example embodiment, a method includes: placing a trocar cannula in an eye of a patient; inserting a surgical instrument into the eye of the patient; imaging the eye of the patient using an imaging device; sensing a location of the trocar cannula using a sensor; and performing by a controller: receiving one or more three-dimensional images from the imaging device; receiving coordinates of the trocar cannula from the sensor; generating a three-dimensional map of the eye from the one or more three-dimensional images and the coordinates, the three-dimensional map including a representation of the trocar cannula; generating guidance for performing an ophthalmic procedure according to the three-dimensional map; and at least one of (a) outputting the guidance to a display device and (b) controlling an actuator coupled to a surgical instrument within the trocar cannula according to the guidance.

In some implementations of the first example embodiment, the imaging device includes both an optical coherence tomography imaging device and a three-dimensional camera.

In some implementations of the first example embodiment, the method includes performing, by the controller: detecting one or more representations of one or more items of anatomy in the three-dimensional map; generating an instrument envelope according to the one or more representations; and detecting regions of the eye traversed by the surgical instrument; generating an instrument path representing portions of the eye remaining to be treated according to the regions and a treatment plan; and at least one of (a) output a representation of the surgical instrument and the instrument path to the display device and (b) control the actuator coupled to the surgical instrument according to the instrument envelope and the instrument path.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A system comprising:
an imaging device configured to perform three-dimensional imaging of at least a portion of an eye of a patient;
a sensor configured to sense a location of a trocar cannula positioned in the eye of the patient; and
a controller configured to:
receive one or more three-dimensional images from the imaging device;
receive coordinates of the trocar cannula from the sensor;
generate a three-dimensional map of the eye from the one or more three-dimensional images and the coordinates, the three-dimensional map including a representation of the trocar cannula;
generate guidance for performing an ophthalmic procedure according to the three-dimensional map; and
at least one of (a) output the guidance to a display device and (b) control an actuator coupled to a surgical instrument within the trocar cannula according to the guidance.

2. The system of claim 1, wherein the imaging device is an optical coherence tomography imaging device.

3. The system of claim 1, wherein the imaging device is a three-dimensional camera.

4. The system of claim 1, wherein the imaging device includes both an optical coherence tomography imaging device and a three-dimensional camera.

5. The system of claim 1, wherein the sensor comprises two or more cameras configured to detect one or more fiducial markers on the trocar cannula.

6. The system of claim 1, wherein the sensor comprises a plurality of local positioning sensors configured to sense signals transmitted from the trocar cannula.

7. The system of claim 1, wherein the sensor comprises a plurality of local positioning sensors configured to sense signals transmitted from one or more radio frequency identifiers (RFID) devices in the trocar cannula.

8. The system of claim 1, wherein the controller is configured to:
detect one or more representations of one or more items of anatomy in the three-dimensional map;
generate an instrument envelope according to the one or more representations; and
at least one of (a) output a representation of the instrument envelope to the display device and (b) control the actuator coupled to the surgical instrument according to the instrument envelope.

9. The system of claim 8, wherein the controller is configured to detect the one or more representations of the one or more items of anatomy using one or more machine learning models.

10. The system of claim 8, wherein the instrument envelope corresponds to a posterior chamber of the eye of the patient.

11. The system of claim 8, wherein the instrument envelope corresponds to an anterior chamber of the eye of the patient.

12. The system of claim 8, wherein the instrument envelope corresponds to an interior of a capsular bag of the eye of the patient.

13. The system of claim 1, wherein the controller is further configured to:
detect regions of the eye traversed by the surgical instrument;
generate an instrument path representing portions of the eye remaining to be treated according to the regions and a treatment plan; and
at least one of (a) output a representation of the instrument path to the display device and (b) control the actuator coupled to the surgical instrument according to the instrument path.

14. The system of claim 13, wherein the controller is configured to generate the instrument path using a machine learning model.

15. The system of claim 1, wherein the controller is configured to perform (b) and the actuator is a robotic arm.

16. The system of claim 15, wherein the ophthalmic procedure defines placement of at least one of incisions and shunts in a trabecular meshwork of the eye for treating glaucoma.

17. The system of claim 15, wherein the ophthalmic procedure defines phacoemulsification of a lens of the eye.

18. The system of claim 15, wherein the ophthalmic procedure defines a vitrectomy of the eye.

19. The system of claim 15, wherein the ophthalmic procedure defines peeling of a membrane.

20. The system of claim 15, wherein the ophthalmic procedure defines performing retinal reattachment.
